(19) United States
(12) Patent Application Publication
(10) Pub. No.: US 2009/0297485 A1
(43) Pub. Date: Dec. 3, 2009

(54) COMPOSITIONS AND METHODS FOR TREATING PSYCHIATRIC AND NEURODEGENERATIVE DISORDERS

(76) Inventor: Allan Mishra, Menlo Park, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/473,915

(22) Filed: May 28, 2009

Related U.S. Application Data
(60) Provisional application No. 61/056,596, filed on May 28, 2008.
(60) Provisional application No. 61/091,266, filed on Aug. 22, 2008.

(51) Int. Cl. A61K 35/12; A61K 35/16; A61P 25/16; A61P 25/00

(52) U.S. Cl. 424/93.7; 424/530

(57) ABSTRACT

Treatment methods are disclosed for psychiatric and neurodegenerative disorders by treatment of the patient with platelet-rich plasma (PRP). PRP is administered to areas of the brain that have been identified as associated with the psychiatric or neurodegenerative disorder to replenish the dysfunctional tissue.

### RELATED APPLICATIONS

This application claims priority to U.S. Provisional Application No. 61/056,596, filed May 28, 2008 and U.S. Provisional application No. 61/091,266, filed August 22, 2008, both of which are incorporated herein by reference in their entirety, including specifications.

### BACKGROUND

1. Field of the Invention

Embodiments of the invention relate to treatment of psychiatric disorders and neurodegenerative disorders (including without limitation depression, amyotrophic lateral sclerosis (ALS), Parkinson's disease, multiple sclerosis, Alzheimer's Disease and stroke) by administration of blood factors, such as platelet-rich plasma, to brain tissue.

2. Description of the Related Art

Platelets are living but terminal cytoplasmic portions of marrow megakaryocytes. They have no nucleus for replication and will die off in 5-9 days. They adhere together to form a platelet plug at an injury site and actively extrude the growth factors involved in initiating wound healing. These growth factors, also called cytokines, are small proteins each of about 25,000 Daltons molecular weight. They are stored in a granules in platelets. In response to platelet to platelet aggregation or platelet to connective tissue contact, the cell membrane of the platelet is “activated” to release these alpha granules. These growth factors include platelet derived growth factors (PDGF), transforming growth factor beta 1 and 2 (TGF-β), fibronectin, vitronectin, fibrin and insulin-like growth factor (ILGF). These growth factors function to assist the body in repairing itself by stimulating stem cells to regenerate new tissue and by promoting vascularization.

A wide variety of cytokines are released by activated platelets. Platelet derived growth factor (PDGF), transforming growth factor-beta (TGF-β), platelet-derived angiogenesis factor (PDAF) and platelet derived endothelial cell growth factor (PD-ECGF) and insulin-like growth factor (IGF) are among the cytokines released by degranulating platelets. These cytokines serve a number of different functions in the healing process, including helping to stimulate cell division and promote vascularization/revascularization at an injury site. They also work as powerful chemotactic factors for mesenchymal cells, monocytes and fibroblasts, among others. For the purposes of this patent, the term “releasate” refers to the internal contents of the platelet, including cytokines, which have the potential to affect another cells' function.

Platelet rich plasma (PRP) is an autologous biologic tool that has emerged as a safe potential adjunctive or stand alone treatment for diverse disorders such as chronic tendonitis (see U.S. Pat. No. 6,811,777 which is incorporated herein by reference in its entirety) and improvement of impaired cardiac function (see U.S. Pat. No. 7,314,617 which is incorporated herein by referencein its entirety). PRP contains growth factors that stimulate the proliferation of a variety of cells as well as serotonin, adenosine and calcium. In response to platelet to platelet aggregation or platelet to connective tissue contact the cell membrane of the platelet is “activated” to secrete the contents of the alpha granules. The alpha granules release cytokines via active secretion through the platelet cell membrane as histones and carbohydrate side chains are added to the protein backbone to form the complete cytokine. Platelet disruption or fragmentation, therefore, does not result in release of the complete cytokine.

Historically, PRP has been used to form a fibrin tissue adhesive through activation of the PRP using thrombin and calcium, as disclosed in U.S. Pat. No. 5,165,938 to Knighton, and U.S. Pat. No. 5,599,558 to Gordinier et al., incorporated in their entirety by reference herein. Activation results in release of the various cytokines and also creates a clotting reaction within various constituents of the plasma fraction. The clotting reaction rapidly forms a platelet gel (PG) which can be applied to various wound surfaces for purposes of hemostasis, sealing, and adhesion.

### SUMMARY

This combination of growth factors and proteins found in PRP may be the ideal treatment for patients with severe depression or neurodegenerative disease that has failed other treatment methods. Platelets are also known to contain a dopamine transporter protein (DAT) that may have significant value for treatment of depression.

Parkinson's disease is a progressive brain disorder characterized by failure of dopaminergic neurons in the substantia nigra of the brain. These cells fail to produce dopamine and the patient experiences symptoms such as loss of motor and speech function in addition to tremors and muscle rigidity. It has been found by the inventor that the components of platelet-rich plasma (PRP) contain many of the growth factors needed to support the cells that are failing in Parkinson's disease. Transforming growth factor beta for example is required for the induction of these cells. Other factors within PRP such as serotonin, adenosine, calcium or even histamine may help retard the progression of the disease or even reverse it. Specifically, PRP may function to stimulate the basal ganglia to produce enough dopamine for a patient to either reduce or eliminate other Parkinson's treatments such as leva dopa.

Depression and other psychiatric disorders cause significant morbidity and even mortality. Traditional treatment methods focus on pharmacologic modifications of neural transmitters and cognitive therapy. Electroshock therapy and even deep brain stimulation have been used but with limited success and significant side effects. A better treatment is needed. Emerging biologic options need to be developed.

Embodiments of the invention are directed to methods of treating a psychiatric disorder or neurodegenerative disorder by administering a composition containing unactivated or activated platelet rich plasma (PRP) to an individual in need thereof. In preferred embodiments, the psychiatric disorder is depression, bipolar disorder or frank psychosis. In preferred embodiments, the neurodegenerative disorder is amyotrophic lateral sclerosis (ALS), Huntington's Chorea, Parkinson's disease, multiple sclerosis, stroke, amyotrophic lateral sclerosis (ALS) or Huntington's Chorea.

In some embodiments, the neurodegenerative disease is stroke and the PRP is administered after the stroke.

In some embodiments, the neurodegenerative disorder is multiple sclerosis and the PRP composition is injected into areas of plaque, whereby trophic factors within PRP remylenate the nerves resulting in improved patient function.

In preferred embodiments, the PRP is buffered to physiological pH prior to administration.

Preferably, the method also includes identifying an area of the brain that is dysfunctional via imaging technology and administering the PRP to that area of tissue. Preferably, the imaging technology is selected from MRI, CT, PET scanning, ultrasound and X-ray.

In preferred embodiments, the PRP is administered via endovascular, extravascular, surgical, stereotactic or robotic guidance to an area of dysfunctional brain tissue. Preferably, PRP is administered to an area of dysfunctional brain tissue using a catheter. Preferably, the PRP is autologous. In some preferred embodiments, the composition for administration also includes stem cells, neural cells and/or neural stem cells. In some preferred embodiments, the composition for administration also includes a PRP extract containing dopamine transporter protein (DAT).

Embodiments of the invention are directed to compositions for the treatment of a psychiatric disorder or neurodegenerative disorder which contain unactivated or activated platelet rich plasma. Preferably, the composition is at physiological pH. Preferably, the composition is autologous. In some preferred embodiments, the composition also includes cells such as neural or stem cells, in particular, neural stem cells. In some embodiments, the composition also includes a PRP extract including dopamine transporter protein (DAT).

Further aspects, features and advantages of this invention will become apparent from the detailed description of the preferred embodiments which follow.

### DETAILED DESCRIPTION OF THE PREFERRED EMBODIMENTS

Embodiments of the invention are directed to methods for treating psychiatric and neurodegenerative disorders by administering a composition that includes unactivated or activated platelet rich plasma (PRP) or an extract thereof (PRP extract) to an individual (human or animal) in need thereof. In preferred embodiments, the psychiatric or neurodegenerative disease is Depression, Bipolar Disorder or frank psychosis. This application is not limited to this list but could be applied in treatment of diverse forms of psychiatric disorders. Neurodegenerative disorders including but not limited to amyotrophic lateral sclerosis (ALS), Huntington's Chorea, Parkinson's disease, multiple sclerosis, and stroke are also treated with PRP. A more comprehensive listing of neurodegenerative disease which may be treated by PRP, PRP releasate and/or PRP extract administered as described herein is found in Table 1 below.

TABLE 1
Neurodegenerative Diseases

```
Adrenal Leukodystrophy (ALD)
    Alcoholism
    Alexander's disease
    Alper's disease
    Alzheimer's disease
    Amyotrophic lateral sclerosis (Lou Gehrig's Disease)
    Ataxia telangiectasia
    Batten disease (also known as Spielmeyer-Vogt-Sjögren-Batten
    disease)
    Bovine spongiform encephalopathy (BSE)
    Canavan disease
    Cerebral palsy
    Cockayne syndrome
    Corticobasal degeneration
    Creutzfeldt-Jakob disease
    Familial Fatal Insomnia
    Frontotemporal lobar degeneration
    Huntington's disease
    HIV-associated dementia
    Kennedy's disease
    Krabbe's disease
    Lewy body dementia
    Neuroborreliosis
    Machado-Joseph disease (Spinocerebellar ataxia type 3)
    Multiple System Atrophy
    Multiple sclerosis
    Narcolepsy
    Niemann Pick disease
    Parkinson's disease
    Pelizaeus-Merzbacher Disease
    Pick's disease
    Primary lateral sclerosis
    Prion diseases
    Progressive Supranuclear Palsy
    Refsum's disease
    Sandhoff disease
    Schilder's disease
    Subacute combined degeneration of spinal cord secondary to
    Pernicious Anaemia
    Spielmeyer-Vogt-Sjogren-Batten disease (also known as Batten
    disease)
    Spinocerebellar ataxia (multiple types with varying characteristics)
    Spinal muscular atrophy
    Steele-Richardson-Olszewski disease
    Tabes dorsalis
    Toxic encephalopathy
```

PRP, PRP releasate and/or PRP extract may be administered by catheter, syringe, or in combination with an implantable device. In preferred embodiments, the PRP, PRP releasate and/or PRP extract is administered via endovascular, extravascular, surgical, stereotactic or robotic guidance to an area of dysfunctional brain tissue using a catheter or other insertion device. In a manner similar to deep brain stimulation, an electrode is placed with image guidance and then the PRP, PRP releasate and/or PRP extract is delivered either alone or in conjunction with this therapy or optionally with electroshock therapy, cells such as neural cells, stem cells or neural stem cells or other drugs. PRP may be combined with biologically derived products such as bone marrow, stems cells or cells from another part of the body.

In some embodiments, PRP, PRP releasate and/or PRP extract is administered to the motor areas of the brain affected by a degenerative disorder such as ALS. The targeted brain areas are identified via clinical symptoms or via imaging techniques or a combination of techniques. The PRP in an activated or unactivated form or an extract thereof is then applied via a single treatment injection or via multiple treatments.

Other neurodegenerative disorders such as Parkinson's and Alzheimer's disease are treated in a similar fashion.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance. Platelet-rich plasma

The term “PRP” is used synonymously with platelet-rich plasma and as used herein, is a broad term which is used in its ordinary sense and is additionally defined for purposes of this application as a concentration of platelets greater than the peripheral blood concentration suspended in a solution of plasma. In some embodiments, the platelets are suspended in an excipient other than plasma or the platelet composition includes other excipients suitable for administration to a human or non-human animal including, but not limited to isotonic sodium chloride solution, physiological saline, normal saline, dextrose 5% in water, dextrose 10% in water, Ringer solution, lactated Ringer solution, Ringer lactate, Ringer lactate solution, and the like. Typically, platelet counts in PRP as defined herein range from 500,000 to 1,200,000 per cubic millimeter, or even more. PRP may be obtained using autologous, allogenic, or pooled sources of platelets and/or plasma. PRP may be obtained from a variety of animal sources, including human sources. In preferred embodiments, PRP according to the invention is buffered to physiological pH.

Platelet-rich plasma (PRP) is obtained from whole blood or plasma by concentrating the platelets from the blood. While whole blood may contain about 95% red blood cells, about 5% platelets and less than 1% white blood cells, PRP may contain 95% platelets with 4% red blood cells and 1% white blood cells. PRP can be combined with activating agents such as thrombin or calcium which activate the platelets to release their contents such as cytokinins and other growth factors. PRP as defined herein comprises unactivated platelets, activated platelets, or the like, or a combination thereof.

In some embodiments, the composition comprises platelet releasate wherein the composition is at a pH greater than or equal to physiological pH, and wherein the composition comprises substantially no unactivated platelets.

In another embodiment, the inventive platelet composition may comprise releasate from platelets, in addition to platelets themselves. The releasate comprises the various cytokines released by degranulating platelets upon activation. Many activators of platelets exist; these include calcium ions, thrombin, collagen, and adenosine diphosphate. Releasates according to the invention may be prepared according to conventional methods, including those methods described in U.S. Pat. No. 5,165,938 to Knighton, and U.S. Pat. No. 5,599,558 to Gordinier et al.

In some preferred embodiments, the PRP composition is supplemented with serotonin, adenosine, and/or calcium salt. In some preferred embodiments, the PRP composition is supplemented with cells such as stem cells.

The platelet composition may be prepared using any conventional method of isolating platelets from whole blood or platelet-containing blood fractions. These include centrifugal methods, filtration, affinity columns, and the like. If the platelet composition comprises PRP, then conventional methods of obtaining PRP, such as those disclosed in U.S. Pat. Nos. 5,585,007 and 5,788,662 both to Antanavich et al., incorporated herein by reference in their entirety, may be utilized. The platelet compositions disclosed herein include PRP, activated and unactivated, as well as platelet realeasate and platelet extracts which are a platelet fraction which has been purified to enrich in specific components such as DAT. Methods of delivery and treatment as described herein are generally applicable to these platelet compositions.

Adjusting the pH of platelet compositions has been used to prolong the storage time of unactivated platelets, as disclosed in U.S. Pat. No. 5,147,776 to Koerner, Jr. and U.S. Pat. No. 5,474,891 to Murphy, incorporated by reference herein in their entirety. pH may be adjusted using a variety of pH adjusting agents, which are preferably physiologically tolerated buffers, but may also include other agents that modify pH including agents that modify lactic acid production by stored platelets. Especially useful are those pH adjusting agents that result in the pH of the platelet composition becoming greater than or equal to physiological pH. In an embodiment, the pH adjustment agent comprises sodium bicarbonate. Physiological pH, for the purposes of this invention, may be defined as being a pH ranging from about 6.5-8.0, more preferably from 7.3 to 7.5, yet more preferably from about 7.35 to about 7.45. pH adjusting agents useful in the practice of this invention include bicarbonate buffers (such as sodium bicarbonate), calcium gluconate, choline chloride, dextrose (d-glucose), ethylenebis(oxyethylenenitrilo)tetraacetic acid (EGTA), monobasic phosphate, 4-(2-hydroxyethyl)-1-piperazineethanesulfonic acid (HEPES), maleic acid, 4-morpholinepropanesulfonic acid (MOPS), 1,4-piperazinebis(ethanesulfonic acid) (PIPES), N-tris(hydroxymethyl)methyl-2-aminoethanesulfonic acid (TES), tris(hydroxymethyl)aminomethane (TRIS BASE), tris(hydroxymethyl)aminomethane hydrochloride (TRIS.HCl), and urea. In a preferable embodiment, the pH adjusting agent is a bicarbonate buffer, more preferably, sodium bicarbonate.

In an aspect, the invention relates to the method wherein the platelet composition is at or above physiological pH. In an aspect, the invention relates to the method wherein the platelet composition optionally includes platelet releasate. In some embodiments, PRP plus an exogenous activator of platelets may be administered to the patient such as thrombin, epinephrine, collagen, or calcium salts. In some embodiments, the platelet composition is substantially free from exogenous activators. In some preferred embodiments of the invention, the platelet composition is administered without an exogenous platelet activator. It is a particular advantage of the invention that administration of PRP without an exogenous activator is effective to treat the disease conditions and psychiatric disorders disclosed herein.

### Dopamine and Dopamine Transporter Protein

Dopamine is a hormone and neurotransmitter occurring in a wide variety of animals, including both vertebrates and invertebrates. In the brain, this phenethylamine functions as a neurotransmitter, activating the five types of dopamine receptors—D1, D2, D3, D4 and D5, and their variants. Dopamine is produced in several areas of the brain and is involved in diverse functions. Increased levels of dopamine are associated with Schizophrenia, and psychosis. Depressed dopamine levels are associated with Parkinsons's Disease, Attention Deficit Disorder (ADD), depression and social anxiety.

The dopamine transporter (also dopamine active transporter, DAT) is a membrane-spanning protein that binds dopamine and is found in platelets. In preferred embodiments of the invention, administration of PRP increases DAT levels and DAT activity and relieves symptoms for the patient suffering from a psychiatric or neurodegenerative disorder, particularly Parkinson's Disease, and severe forms of depression, ADD, and social anxiety. DAT provides the primary mechanism through which dopamine is cleared from synapses, transporting dopamine from the synapse into a neuron. DAT is present in the peri-synaptic area of dopaminergic neurons in areas of the brain where dopamine signaling is common. Because DAT terminates the dopamine signal, it is implicated in a number of dopamine-related disorders, including Schizophrenia, psychosis, Parkinsons's Disease, Attention Deficit Disorder, social anxiety, clinical depression, and alcoholism. Additionally, decreasing levels of DAT expression are associated with aging, and likely underlie a compensatory mechanism for the decreases in dopamine release as a person ages (Bannon M J, et al. “Dopamine transporter mRNA content in human substantia nigra decreases precipitously with age”. Proc. Natl. Acad. Sci. U.S.A. 89 (15): 7095-9).

In some embodiments, platelet rich plasma is used as a source material for further purification of platelet components such as DAT (PRP extract). Alternatively, DAT may be isolated from other source materials or produced as a recombinant protein. The human gene for DAT has been described (Kawarai T, Kawakami H, Yamamura Y, Nakamura S (1997). “Structure and organization of the gene encoding human dopamine transporter”. Gene 195 (1): 11-18) and methods of protein isolation are known in the art. The DAT preparation may be partially purified or purified to homogeneity as determined by SDS-PAGE. In preferred embodiments, the DAT is autologous. Platelet-derived DAT is used to treat one or more of the neurodegenerative disorders by administration using the same methods as described above for PRP. Other platelet-derived proteins obtainable from PRP may be isolated or partially purified for treatment and/or prevention of specific disorders.

### Delivery to Brain Tissue

In preferred embodiments, delivery of PRP, PRP releasate and/or PRP extract is to areas of the brain associated with dopamine transmission. While there are eight dopaminergic pathways, the four major ones are: mesolimbic pathway, mesocortical pathway, nigrostriatal pathway, and tuberoinfundibular pathway.

The mesolimbic pathway transmits dopamine from the ventral tegmental area (VTA) to the nucleus accumbens. The VTA is located in the midbrain, and the nucleus accumbens in the limbic system. The mesocortical pathway transmits dopamine from the VTA to the frontal cortex. Malfunctions of the mesocortical pathway are associated with schizophrenia. The nigrostriatal pathway transmits dopamine from the substantia nigra to the striatum. This pathway is associated with motor control, and degeneration of this pathway is related to Parkinson's disease. The tuberoinfundibular pathway transmits dopamine from the hypothalamus to the pituitary gland. This pathway influences the secretion of certain hormones, including prolactin. The neurons of the dopaminergic pathways have axons which run the entire length of the pathway. The neuron's soma produces the dopamine, which is then transmitted via the projecting axons to their synaptic destinations.

Administration may be a single or repeated administrations. The administration may be continuous or for a period of time. Administration may be at a single site or at multiple sites. The amount of PRP and/or platelet releasate to be administered will vary depending upon the manner of administration, the age, sex, condition and body weight of the patient, and with the type of disease, and size of the patient predisposed to or suffering from the disease. Generally, a dosage comprising 1 to 5 million platelets is adequate. Typically, 3-5 cc of pH-adjusted PRP is administered at one or multiple sites. More preferably, 2-4 cc is administered. In preferred embodiments, the PRP, PRP releasate and/or extract of PRP is autologous. Delivery may be accomplished by means known in the art such as catheter, shunt, syringe, stent, or topical delivery.

### Extravascular

In some embodiments, administration may be extravascular including intramuscular, subcutaneous, and intrathecal. Intrathecal injection may be one or more intracisternal injection(s) into the caudal region of the brain. The PRP, PRP releasate and/or PRP extract may be delivered after surgically accessing an area to be treated.

### Endovascular

Endovascular administration may be either intravenous or intra-arterial. Endovascular surgery is a form of minimally invasive surgery by which a region of the body may be accessed by a major blood vessel. In preferred embodiments, a catheter or other insertion device is introduced percutaneously into a large blood vessel, such as the femoral artery for administration of PRP-containing compositions of the invention.

Stereotactic surgery or stereotaxy is a minimally-invasive form of surgical intervention which makes use of a three-dimensional coordinates system to locate small targets inside the body, particularly in the brain. In some embodiments, stereotaxy is used to locate optimal locations in the brain for administration of PRP, PRP releasate and/or PRP extract.

In some embodiments, the stereotactic technique may include robotic guidance. See for example U.S. Pat. No. 5,735,278 which is incorporated herein by reference in its entirety. One device which may be used for robotic guidance during neurosurgery is the NeuroArm® which is a surgical robot specifically designed for neurosurgery. It may be image-guided and can perform procedures inside an MRI.

### Imaging Techniques

Commonly used imaging methods include, but are not limited to MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging and ultrasound techniques.

### Stem Cells

Stem cells are undifferentiated cells which have the capacity to develop into any or differentiated cell types. Stem cells and their progeny progenitor cells act as a repair system for the body, replenishing specialized cells. In some preferred embodiments of the invention, stem cells are included with the PRP compositions according to the invention for their ability to replenish tissue that is unhealthy or dysfunctional. Preferably, the stem cells are autologous. In some preferred embodiments, the stem cells are neural stem cells.

### Diagnostic Applications

PRP, PRP releasate and/or PRP extract may be used as a diagnostic tool for a brain disorder or other disease. Levels of PRP components may be altered in specific disease states. The levels of one or more components of PRP from a disease population are determined and compared to normative values for specific components of PRP. An individual presenting with symptoms characteristic of a neurodegenerative disorder is then tested for components of PRP known to vary in diseased populations. The levels of PRP components are compared to values from a normal population.

Alternatively, the effect of PRP, PRP releasate and/or PRP extract on neural stem cells which are models for neurodegenerative diseases including Parkinson's disease and stroke will be evaluated. ReNcell® (VM Neural Stem Cell Line; Millipore), a human neural progenitor line, is a model system for Parkinson's Disease which is useful for the study of treatment options for Parkinson's disease, including PRP, PRP releasate and/or PRP extract.

Cell Culture with PRP for Treatment of Neurodegenerative and Psychiatric Disorders

PRP is obtained from a patient which may be any animal, mammal, or human and formulated as PRP, PRP releasate and/or PRP extract. The formulation is used to form a cell culture medium which in turn is used to grow cells. The cells or products such as proteins produced by the cells are used to create a formulation which is administered to a patient to treat the patient. In some preferred embodiments, the formulation includes the DAT protein. The patient treated may be the same patient from which the PRP, PRP releasate and/or PRP extract and/or the cells are obtained. In addition to cells, tissue such as neural tissue may be cultured on the medium and the tissue used to treat a patient, particularly the patient the tissue was taken from.

A cell culture medium of the invention may consist only of platelets, PRP or treated PRP. However, the medium may be a conventional medium supplemented with platelets, PRP, platelet releasate or combinations thereof. In one embodiment the platelets are concentrated and subjected to treatment (e. g. sonication) whereby the platelets are caused to break open and provide a releasate. The releasate is used to formulate the culture medium upon which the cells or tissue are cultured. The cells or tissue are maintained on the culture medium under conditions which promote cell growth and proliferation. The cells produced are used to create a formulation. The formulation is administered to the patient to treat a disease. Alternatively, tissue such as neural tissue grown on the medium is used to treat the patient. The medium may comprise a cell assimilable source of carbon of carbon, nitrogen, amino acids, iron, inorganic ions, and trace elements.

Media formulations are generally prepared according to methods known in the art. Accordingly, any standard medium, e. g., RMPI-1630 Medium, CMRL Medium, Dulbecco's Modified Eagle Medium (D-MEM), Fischer's Medium, Iscove's Modified Dulbecco's Medium, McCoy's Medium, Minimum Essential Medium, NCTC Medium, and the like can be formulated with PRP or platelet releasate at the desired effective concentration. If desired, media supplements, e. g., salt solutions (e. g., Hank's Balanced Salt Solution or Earle's Balanced Salt Solution), antibiotics, nucleic acids, amino acids, carbohydrates, and vitamins are added according to known methods. If desired, growth factors, colony-stimulating factors, cytokines and the like can also be added to media according to standard methods. For example, media of the invention can contain any of the following substances, alone or in combination, with PRP or platelet releasate and/or extract thereof: erythropoietin, granulocyte/macrophage colony-stimulating factor (GM-CSF), granulocyte colony-stimulating factor (G-CSF), macrophage colony-stimulating factor (M-CSF), an interleukin (e. g., IL-1, IL-2, IL-3, IL-4, IL-5, etc. ), insulin-growth factor (IGF), transferrin, albumin, and stem-cell growth factor (SCF).

If desired, identification and separation of expanded subpopulations of cells is performed according to standard methods. For example, cells may be analyzed by fluorescence-activated cell sorting (FACS). This procedure generally involves labeling cells with antibodies coupled to a fluorescent dye and separating the labeled cells from the unlabelled cells in a FACS, e. g., FACScan (Becton Dickson). Thus, virtually any cell can be identified and separated, e. g., by analyzing the presence of cell surface antigens (see e. g., Shah et al., J. Immunol. 140: 1861,1988). When a population of cells is obtained, it is then analyzed biochemically or, alternatively, provides a starting population for additional cell culture, allowing the action of the cells to be evaluated under defined conditions in culture.

The therapeutic method (s) and compositions of the present invention may also include co-administration with other human growth factors. Exemplary cytokines or hematopoietins for such use include, without limitation, factors such as an interleukin (e. g., IL-1), GM-CSF, G-CSF, M-CSF, tumor necrosis factor (TNF), transferrin, and erythropoietin. Growth factors like B cell growth factor, B cell differentiation factor, or eosinophil differentiation factors may also prove useful in co-administration with PRP, PRP extract and/or releasate. The dosage recited above would be adjusted to compensate for such additional components in the therapeutic composition. Progress of the treated patient can be monitored by conventional methods.

Once grown on a cell culture medium of the invention the cells or products produced from the cells can be formulated into a pharmaceutically acceptable formulation and administered to a patient which may be a human patient and may be the same human patient from which the platelets and/or the cells were derived.

In Vitro/Ex Vivo Assays to Determine Effectiveness of PRP, PRP Releasate and/or PRP Extract in Disease Treatment

PRP, PRP releasate and/or PRP extract may also be specifically used as a neurodegenerative disease tool by growing an individual's brain or neural cells in culture with PRP, PRP releasate and/or PRP extract. If they are found to grow well as determined by specific molecular markers it will be possible to predict the severity of a specific disorder or disease state.

Cells obtained from an individual with symptoms of a neurodegenerative disease or disorder are obtained and cultured. PRP, PRP releasate and/or PRP extract is added to the cell culture of diseased cells to evaluate potential efficacy in treatment, prevention and/or amelioration of disease symptoms.

### EXAMPLES

### Example 1

PRP was prepared using a centrifuge unit made by Harvest (Plymouth, Mass.). (Similar units are available as The Biomet GPS™ system, the Depuy Symphony™ machine and the Medtronic Magellan™, Arthrex ACP™, blood bank device or other PRP machine.) Blood (1-500 cc or more) was drawn from the patient using a standard sterile syringe, combined with 5 cc of a citrate dextrose solution for anticoagulation, and then spun down to isolate the platelets according to the manufacturer's protocol. These platelets were then resuspended in approximately 3 cc of plasma. The resulting platelet rich plasma solution (PRP) was quite acidic and was neutralized with using approximately 0.05 cc of an 8.4% sodium bicarbonate buffer per cc of PRP under sterile conditions to approximately physiologic pH of 7.4. The PRP was not activated through addition of exogenous activators. This PRP composition is referred to herein as autologous platelet extract (APEX).

### Example 2

Fifty cc of whole blood is drawn from a patient, and then prepared according to the method of Knighton, U.S. Pat. No. 5,165,938, column 3. The PRP is activated according to Knighton using recombinant human thrombin. The degranulated platelets are spun down and the releasate containing supernatant is recovered. The releasate may be optionally pH adjusted to a pH of 7.4 using sodium bicarbonate buffer.

### Example 3

Thirty ml of whole blood were drawn from a patient. A platelet composition was prepared according to Example 1 of U.S. Pat. No. 5,510,102 to Cochrum, incorporated herein by reference in its entirety, except that no alginate is added to the platelet composition.

### Example 4

### Administration to Brain Tissue

A patient presents with symptoms of a psychiatric or neurodegenerative disorder. An area of the brain associated with the disorder is identified as the target area. Using the technique of Example 1, an autologous platelet extract (APEX) is obtained and buffered to physiologic pH.

The target area is located by stereotactic technique. APEX is then introduced into the target area using a catheter. This could be done under local or general anesthesia and with or without imaging guidance.

### Example 5

### Treatment of Cells to Prevent Apoptosis

Mammalian cells, such as brain cells, neural cells or supportive vascular endothelial cells, are cultured using appropriate culture media under low oxygen tension to provide conditions of oxidative stress and simulate apoptosis. The cells are normal or diseased. The PRP is added to the culture media to evaluate the effect of PRP on apoptosis. The effective concentration for administration of PRP to cells is determined by testing increasing concentrations of PRP in the culture media under the conditions of oxidative stress and compared to cells cultured in media which does not contain PRP. Test conditions include administration of PRP before and/or after application of oxidative stress.

The effect of PRP on cell apoptosis is evaluated using markers of apoptosis such as but not limited to cleaved PARP, caspase-9, procaspase 9, Bax and Bcl-2 measured at predetermined time points such as 6, 12 and 24 hours. Western blot analysis, gene chip analysis and or other means would be done to determine how well PRP prevented and/or treated apoptosis. The ability of PRP to treat or prevent conditions of low oxygen tension is predictive of effectiveness of PRP in treatment of disorders where tissue is under low oxygen tension such as stroke.

### Example 6

### Treatment of Traumatic Brain Disorder or Stroke

PRP is prepared as described above for treatment of a patient with a traumatic brain disorder or stroke. Specifically, a patient who has sustained a closed or open head injury is treated with PRP via an endovascular, stereotactic or other minimally invasive or surgical manner to improve the function of their brain tissue. A patient may be treated with PRP alone or in combination with a hydrogel. The area of damaged brain tissue is identified with imaging and then delivered via one of the means outlined herein including but not limited to endovascular, transvascular, endoscopic or open surgical techniques.

### Example 7

### Treatment with Cells Cultured with PRP

Using the technique of Example 1, an autologous platelet extract (APEX) is obtained and buffered to physiologic pH. Neural cells are then isolated from the patient and grown in a media rich in the APEX in various conditions and dilutions. The APEX promotes cell differentiation and production of proteins such as collagen. The APEX may augment or promote the ability of the cells to transform into non-diseased cells. The cultured cells are monitored to evaluate conversion to a non-disease phenotype. Cells that display phenotypically normal characteristics are reintroduced back into the patient after culture with buffered APEX.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A method of treating a psychiatric disorder or neurodegenerative disorder which comprises administering a composition comprising unactivated or activated platelet rich plasma (PRP) to an individual in need thereof.

2. The method of claim 1, wherein the psychiatric disorder is selected from the group consisting of depression, bipolar disorder and frank psychosis.

3. The method of claim 1, wherein the neurodegenerative disorder is Parkinson's disease, multiple sclerosis, stroke, amyotrophic lateral sclerosis (ALS) or Huntington's Chorea.

4. The method of claim 3, wherein the neurodegenerative disease is stroke and the PRP is administered after the stroke.

5. The method of claim 3, wherein the neurodegenerative disorder is multiple sclerosis and which comprises injecting the PRP composition into areas of plaque, whereby trophic factors within PRP remylenate the nerves resulting in improved patient function.

6. The method of claim 1, wherein the PRP is buffered to physiological pH prior to administration.

7. The method of claim 1, further comprising identifying an area of the brain that is dysfunctional via imaging technology and administering the PRP to that area of tissue.

8. The method of claim 7, wherein the imaging technology is selected from the group consisting of MRI, CT, PET scanning, Ultrasound and X-ray.

9. The method of claim 1, wherein the PRP is administered via endovascular, extravascular, surgical, stereotactic or robotic guidance to an area of dysfunctional brain tissue.

10. The method of claim 1, wherein PRP is administered to an area of dysfunctional brain tissue using a catheter.

11. The method of claim 1, wherein the PRP is autologous.

12. The method of claim 1, wherein the composition further comprises stem cells, neural cells and/or neural stem cells.

13. The method of claim 1, wherein the composition further comprises a PRP extract comprising dopamine transporter protein (DAT).

14. A composition for the treatment of a psychiatric disorder or neurodegenerative disorder comprising unactivated or activated platelet rich plasma.

15. The composition of claim 14, wherein the composition is at physiological pH.

16. The composition of claim 14 which is autologous.

17. The composition of claim 14, further comprising neural cells or stem cells.

18. The composition of claim 17, wherein the stem cells are neural stem cells.

19. The composition of claim 14, which further comprises a PRP extract comprising dopamine transporter protein (DAT).
